(19) United States
(12) Patent Application Publication
(10) Pub. No.: US 2021/0032417 A1
(43) Pub. Date: Feb. 4, 2021

(54) BORIC ACID-CONTAINING POLYVINYL ALCOHOL NANOPARTICLE AND PREPARATION METHOD THEREOF

(71) Applicant: National Tsing Hua University, Hsinchu City (TW)

(72) Inventors: Fan-Gang Tseng, Hsinchu City (TW); Han-Lin Cho, Hsinchu City (TW); Wei-Jen Chan, Hsinchu City (TW)

(73) Assignee: National Tsing Hua University, Hsinchu City (TW)

(21) Appl. No.: 16/711,463

(22) Filed: Dec. 12, 2019

(30) Foreign Application Priority Data
Aug. 2, 2019 (TW) 108127512

(51) Int. Cl. C08J 3/24; A61K 41/00; C08J 3/21

(52) U.S. Cl. CPC C08J 3/24; B82Y 40/00; C08J 3/21; A61K 41/0095

(57) ABSTRACT

Provided are a boric acid-containing polyvinyl alcohol nanoparticle and a preparation method thereof. The boric acid-containing polyvinyl alcohol nanoparticle includes a polyvinyl alcohol nanoparticle and a boric acid, wherein the boric acid is crosslinked to the polyvinyl alcohol nanoparticle by covalent bonds.

### CROSS-REFERENCE TO RELATED APPLICATION

This application claims the priority benefit of Taiwan application serial no. 108127512, filed on Aug. 2, 2019. The entirety of the above-mentioned patent application is hereby incorporated by reference herein and made a part of this specification.

### BACKGROUND OF THE INVENTION

### Field of the Invention

The invention relates to a polymer nanoparticle and a preparation method thereof, and in particular to a boric acid-containing polyvinyl alcohol nanoparticle and a preparation method thereof.

### Description of Related Art

Boron neutron capture therapy (BNCT) is an important anticancer therapy. To date, sodium borocaptate (BSH) and boronophenylalanine (BPA) have been used in clinical trials for BNCT. However, the two drugs are low in yield, expensive, and have limited characteristics in selectively accumulating in the tumor.

### SUMMARY OF THE INVENTION

The invention provides a boric acid-containing polyvinyl alcohol nanoparticle, wherein boric acid is crosslinked to a polyvinyl alcohol particle by covalent bonds.

The invention provides a preparation method of a boric acid-containing polyvinyl alcohol nanoparticle used for preparing the boric acid-containing polyvinyl alcohol nanoparticle.

The boric acid-containing polyvinyl alcohol nanoparticle of the invention includes a polyvinyl alcohol particle and a boric acid, wherein the boric acid is crosslinked to the polyvinyl alcohol particle by covalent bonds.

In an embodiment of the boric acid-containing polyvinyl alcohol nanoparticle of the invention, a particle size of the boric acid-containing polyvinyl alcohol nanoparticle is between 5 nm and 250 nm.

In an embodiment of the boric acid-containing polyvinyl alcohol nanoparticle of the invention, based on a total weight of the boric acid-containing polyvinyl alcohol nanoparticle, a content of boron atoms is between 1 wt. % and 20 wt. %.

A preparation method of a boric acid-containing polyvinyl alcohol nanoparticle of the invention includes the following steps. A polyvinyl alcohol aqueous solution is provided. A non-aqueous solvent is added to the polyvinyl alcohol aqueous solution to facilitate a cononsolvency effect between the non-aqueous solvent and water in the polyvinyl alcohol aqueous solution to obtain a solution containing a polyvinyl alcohol nanoparticle. A boric acid is added to the solution containing the polyvinyl alcohol nanoparticle.

In an embodiment of the preparation method of the boric acid-containing polyvinyl alcohol nanoparticle of the invention, the non-aqueous solvent includes methanol, ethanol, dimethyl sulfoxide (DMSO), or acetone.

In an embodiment of the preparation method of the boric acid-containing polyvinyl alcohol nanoparticle of the invention, an average molecular weight of a polyvinyl alcohol in the polyvinyl alcohol aqueous solution is not more than 300000.

In an embodiment of the preparation method of the boric acid-containing polyvinyl alcohol nanoparticle of the invention, a dropwise rate of adding the non-aqueous solvent to the polyvinyl alcohol aqueous solution is 4 ml/min to 6 ml/min.

In an embodiment of the preparation method of the boric acid-containing polyvinyl alcohol nanoparticle of the invention, after the non-aqueous solvent is added to the polyvinyl alcohol aqueous solution and before the boric acid is added to the solution containing the polyvinyl alcohol nanoparticle, the solution containing the polyvinyl alcohol nanoparticle is cooled to between 4° C. and 12° C.

In an embodiment of the preparation method of the boric acid-containing polyvinyl alcohol nanoparticle of the invention, a concentration of the non-aqueous solvent is between 0.5 vol. % and 50 vol. %.

In an embodiment of the preparation method of the boric acid-containing polyvinyl alcohol nanoparticle of the invention, a concentration of the polyvinyl alcohol aqueous solution is between 0.01 g/100 ml and 10 g/100 ml.

Based on the above, in the boric acid-containing polyvinyl alcohol nanoparticle of the invention, boric acid is crosslinked to the polyvinyl alcohol particle by covalent bonds, so that boric acid may be stably attached to the polyvinyl alcohol nanoparticle. Therefore, the boric acid-containing polyvinyl alcohol nanoparticle of the invention may have a low boric acid leakage rate and thus has a stable structure and characteristics. When the boric acid-containing polyvinyl alcohol nanoparticle of the invention is applied to BNCT, the drug may be ensured to not be rapidly degraded after being injected into the human body and entering normal cells, and the incidence of side effects may be effectively reduced, and efficacy and safety may be effectively improved.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a perspective view of a boric acid-containing polyvinyl alcohol nanoparticle according to an embodiment of the invention.

FIG. 2 is a flowchart of the steps of a preparation method of a boric acid-containing polyvinyl alcohol nanoparticle according to an embodiment of the invention.

FIG. 3 is a graph of the relationship between boric acid leakage rate and time of a boric acid-containing polyvinyl alcohol nanoparticle according to an embodiment of the invention.

### DESCRIPTION OF THE EMBODIMENTS

Embodiments are provided hereinafter and described in detail with reference to figures. However, the embodiments provided are not intended to limit the scope of the disclosure. Moreover, the figures are only descriptive and are not drawn to scale. For ease of explanation, the same devices below are provided with the same reference numerals.

Terms such as “contain”, “include”, and “have” used in the specification are all open terms, i.e., “contains, but not limited to”.

In the following embodiments, the quantities and shapes mentioned are only used to specifically describe the invention to facilitate the understanding of the content thereof, and not to limit the invention.

In a boric acid-containing polyvinyl alcohol nanoparticle of the invention, boric acid is crosslinked to a polyvinyl alcohol nanoparticle by covalent bonds and stably attached to the polyvinyl alcohol nanoparticle, so that the leakage rate of boric acid may be greatly reduced, and therefore a stable structure and characteristics are achieved. Therefore, when the boric acid-containing polyvinyl alcohol nanoparticle of the invention is applied to boron neutron capture therapy (BNCT), the drug may be ensured to not be rapidly degraded after being injected into the human body and entering normal cells, and the incidence of side effects may be effectively reduced, and efficacy and safety may be effectively improved. Further, the boric acid-containing polyvinyl alcohol nanoparticle of the invention has a simple structure and is easy to prepare, and thus has the advantage of low cost.

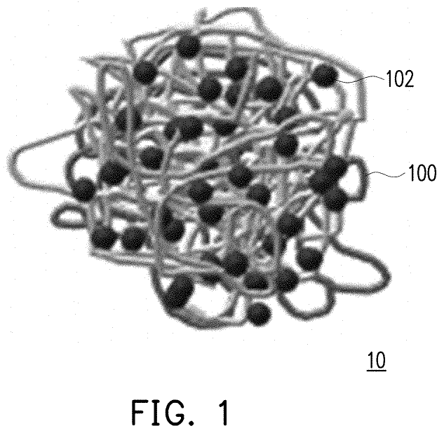

FIG. 1 is a perspective view of a boric acid-containing polyvinyl alcohol nanoparticle according to an embodiment of the invention. Referring to FIG. 1, a boric acid-containing polyvinyl alcohol nanoparticle 10 of an embodiment of the invention includes a polyvinyl alcohol particle 100 and a boric acid 102. The boric acid 102 is crosslinked to the polyvinyl alcohol particle 100 by covalent bonds. In detail, after the boric acid 102 is attached to the polyvinyl alcohol nanoparticle by the covalent bonds, three crosslinking structures may be formed as shown in the following formula 1 to formula 3.

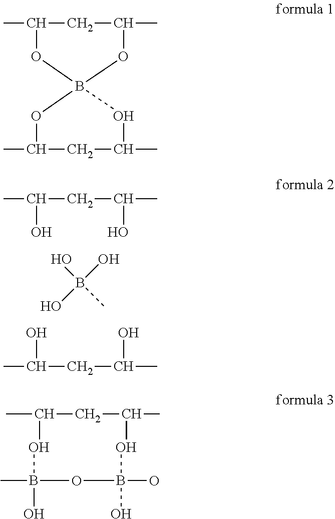

When the boric acid-containing polyvinyl alcohol nanoparticle 10 has any of the above structures or a combination thereof, the boric acid-containing polyvinyl alcohol nanoparticle 10 may have a stable structure, and the boric acid 102 is not readily detached from the polyvinyl alcohol particle 100. Therefore, the boric acid-containing polyvinyl alcohol nanoparticle 10 may have stable and high boron atom content.

In the present embodiment, the boron atom content in the boric acid-containing polyvinyl alcohol nanoparticle 10 is between 1 wt. % and 20 wt. %, based on the total weight of the boric acid-containing polyvinyl alcohol nanoparticle 10.

That is, the boric acid-containing polyvinyl alcohol nanoparticle 10 has high boron content and has better efficacy. Further, in the present embodiment, the particle size of the boric acid-containing polyvinyl alcohol nanoparticle 10 is between 5 nm and 250 nm. By providing the boric acid-containing polyvinyl alcohol nanoparticle 10 at different particle sizes, efficacies may be produced for different symptoms. When the particle size of the boric acid-containing polyvinyl alcohol nanoparticle 10 is within the above range, the boric acid-containing polyvinyl alcohol nanoparticle 10 may be accumulated in the tumor tissue via an enhanced permeability and retention (EPR) effect to improve the efficacy, and the above particle size range is also the particle size range most suitable for driving cancer cells for pinocytosis.

Hereinafter, a preparation method of the boric acid-containing polyvinyl alcohol nanoparticle of the invention is described.

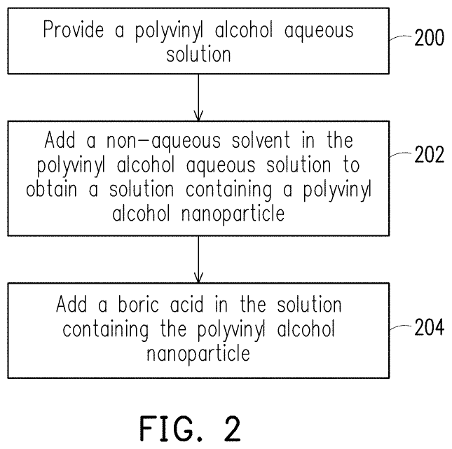

FIG. 2 is a flowchart of the steps of a preparation method of a boric acid-containing polyvinyl alcohol nanoparticle according to an embodiment of the invention. Referring to FIG. 2, first, in step 200, a polyvinyl alcohol aqueous solution is provided. The polyvinyl alcohol aqueous solution is prepared, for example, by adding solid polyvinyl alcohol to deionized distilled water (DD water). In the present embodiment, the concentration of the polyvinyl alcohol aqueous solution is, for example, between 0.01 g/100 ml and 10 g/100 ml. After the solid polyvinyl alcohol is added to the DD water, heating and stirring may be performed, and the temperature is maintained between 70° C. and 90° C. to dissolve the solid polyvinyl alcohol in the DD water. In the present embodiment, the average molecular weight of the polyvinyl alcohol is, for example, not more than 300000.

Next, in step 202, a non-aqueous solvent is added to the polyvinyl alcohol aqueous solution to facilitate a cononsolvency effect between the non-aqueous solvent and water in the polyvinyl alcohol aqueous solution to obtain a solution containing a polyvinyl alcohol nanoparticle. At this time, the solution formed by the non-aqueous solvent and water in the polyvinyl alcohol aqueous solution is not a solvent of polyvinyl alcohol (that is, polyvinyl alcohol is difficult to be dissolved in the formed solution), and thus a solution containing the polyvinyl alcohol nanoparticle is obtained. The non-aqueous solvent is, for example, methanol, ethanol, dimethyl sulfoxide, or acetone.

In the present embodiment, the concentration of the non-aqueous solvent is, for example, between 0.5 vol. % and 50 vol. %.

In detail, when the non-aqueous solvent is added to the polyvinyl alcohol aqueous solution, there is a considerable force between the —OH group of the non-aqueous solvent and the —OH group of water, and the force is greater than the force between polyvinyl alcohol and the non-aqueous solvent and the force between polyvinyl alcohol and water, such that polyvinyl alcohol is insoluble in the solution formed by the non-aqueous solvent and water, which is a so-called cononsolvency effect. Further, polyvinyl alcohol molecules insoluble in the solution formed by the non-aqueous solvent and water form a nanoparticle via the interaction of boronic ester in boric acid and —OH groups of polyvinyl alcohol molecules.

Further, since the cononsolvency effect is rapidly generated after the non-aqueous solvent is added to the polyvinyl alcohol aqueous solution, the rate of addition of the non-aqueous solvent is quite important. In the present embodiment, the dropwise rate when the non-aqueous solvent is added to the polyvinyl alcohol aqueous solution is, for example, between 4 ml/min and 6 ml/min. When the dropwise rate is less than 4 ml/min, the particle size of the formed nanoparticle is greater than 250 nm, and the particle size is increased as the dropwise rate is slowed, so that the nanoparticle may not be accumulated in the tumor tissue via EPR effect and also is not readily phagocytized and is not suitable for tumor treatment. When the dropwise rate is greater than 6 ml/min, the formed nanoparticle has a particle size of less than 5 nm, and thus is not readily phagocytosed and is not suitable for tumor treatment. The size and number of the obtained polyvinyl alcohol nanoparticle may be controlled by changing the dropwise rate.

Then, after the non-aqueous solvent is added to the polyvinyl alcohol aqueous solution, the temperature may be optionally lowered to, for example, between 4° C. and 12° C. When the temperature falls within the above range, the polyvinyl alcohol nanoparticle may be better formed into a spherical particle.

Next, in step 204, boric acid is added to the solution containing the polyvinyl alcohol nanoparticle. In the present embodiment, solid boric acid is added to the solution containing the polyvinyl alcohol nanoparticle, but the invention is not limited thereto. In other embodiments, a boric acid aqueous solution may also be added to the solution containing the polyvinyl alcohol nanoparticle. After boric acid is added to the solution containing the polyvinyl alcohol nanoparticle, boric acid is attached to the polyvinyl alcohol nanoparticle by covalent bonds to form the boric acid-containing polyvinyl alcohol nanoparticle of the invention. In particular, the amount of boric acid that each polyvinyl alcohol nanoparticle may bond depends on its own molecular structure, so the addition of excess boric acid does not increase the boron content of the boric acid-containing polyvinyl alcohol nanoparticle of the invention.

Hereinafter, the boric acid-containing polyvinyl alcohol nanoparticle of the invention is described by experimental examples.

Preparation of polyvinyl alcohol aqueous solution

6 g of polyvinyl alcohol (average molecular weight was 9500) was added to 300 ml of deionized distilled water, and heating and stirring were continued for 2 hours (heating temperature was 75° C., stirring rate was 260 rpm). Next, stirring was performed at a temperature of 25° C. for 12 hours (stirring rate was 260 rpm) to obtain a polyvinyl alcohol aqueous solution having a concentration of about 2 wt. %.

Preparation of solution containing polyvinyl alcohol nanoparticle

15.8 ml of dimethyl sulfoxide (concentration was 5 vol. %) was added to the prepared polyvinyl alcohol aqueous solution, and stirring was performed at a temperature of 25° C. for 1 hour (stirring rate was greater than 360 rpm). Next, the temperature was lowered to 10° C. and maintained for 48 hours to obtain a solution containing the polyvinyl alcohol nanoparticle.

Preparation of Boric Acid-Containing Polyvinyl Alcohol Nanoparticle

3.5 g of boric acid was added to the prepared solution containing the polyvinyl alcohol nanoparticle, and stirring was performed continuously at a temperature of 10° C. for 6 hours (stirring rate was greater than 360 rpm). Then, dialysis was performed with a dimethyl sulfoxide aqueous solution (concentration was 5 vol. %) (using a dialysis membrane having a pore size of 4000 Da) for 24 hours to obtain a boric acid-containing polyvinyl alcohol nanoparticle (particle size between 5 nm and 250 nm). At this point, the amount of boron atoms was about 7.83 wt. %.

Boric Acid Leakage Rate of Boric Acid-Containing Polyvinyl Alcohol Nanoparticle

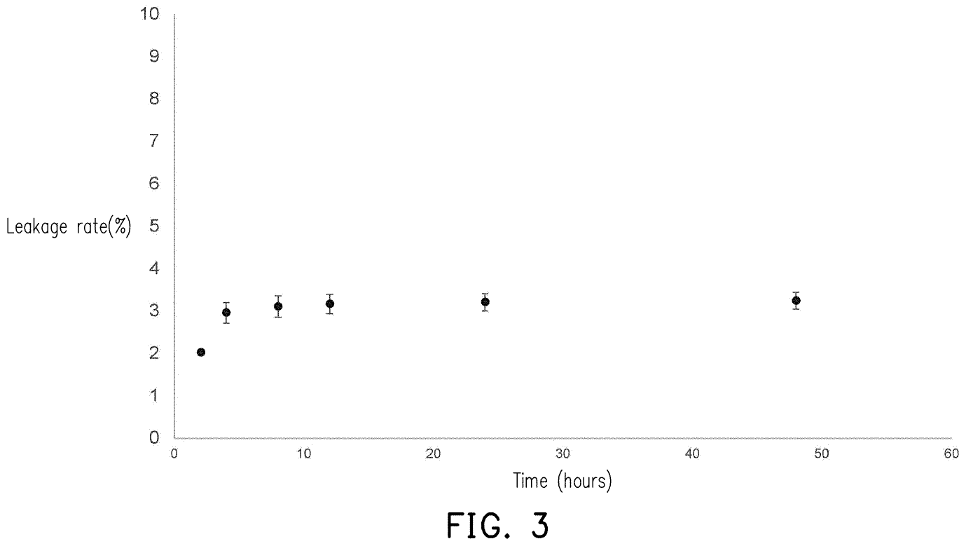

A solution containing the boric acid-containing polyvinyl alcohol nanoparticle of the invention was placed in a dialysis membrane bag having a pore size of 4500 Da, and the dialysis membrane bag was placed in double distilled water for 48 hours, and the results are shown in FIG. 3. As may be seen from FIG. 3, in the boric acid-containing polyvinyl alcohol nanoparticle of the invention, since boric acid was stably attached to the polyvinyl alcohol nanoparticle by covalent bonds, a low boric acid leakage rate (2% to 4%) may be achieved. Further, the boric acid leakage rate of the boric acid-containing polyvinyl alcohol nanoparticle of the invention was not increased with time. Therefore, the boric acid-containing polyvinyl alcohol nanoparticle of the invention may be stably accumulated in tumor tissues to facilitate application in BNCT.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A boric acid-containing polyvinyl alcohol nanoparticle, comprising a polyvinyl alcohol particle and a boric acid, wherein the boric acid is crosslinked to the polyvinyl alcohol particle by covalent bonds.

2. The boric acid-containing polyvinyl alcohol nanoparticle of claim 1, wherein a particle size of the boric acid-containing polyvinyl alcohol nanoparticle is between 5 nm and 250 nm.

3. The boric acid-containing polyvinyl alcohol nanoparticle of claim 1, wherein based on a total weight of the boric acid-containing polyvinyl alcohol nanoparticle, a content of boron atoms is between 1 wt. % and 20 wt. %.

4. A preparation method of a boric acid-containing polyvinyl alcohol nanoparticle, comprising:
providing a polyvinyl alcohol aqueous solution;
adding a non-aqueous solvent to the polyvinyl alcohol aqueous solution to facilitate a cononsolvency effect between the non-aqueous solvent and a water in the polyvinyl alcohol aqueous solution to obtain a solution containing a polyvinyl alcohol nanoparticle; and
adding a boric acid to the solution containing the polyvinyl alcohol nanoparticle.

5. The preparation method of the boric acid-containing polyvinyl alcohol nanoparticle of claim 4, wherein the non-aqueous solvent comprises methanol, ethanol, dimethyl sulfoxide, or acetone.

6. The preparation method of the boric acid-containing polyvinyl alcohol nanoparticle of claim 4, wherein an average molecular weight of a polyvinyl alcohol in the polyvinyl alcohol aqueous solution is not more than 300000.

7. The preparation method of the boric acid-containing polyvinyl alcohol nanoparticle of claim 4, wherein a dropwise rate of adding the non-aqueous solvent to the polyvinyl alcohol aqueous solution is 4 ml/min to 6 ml/min.

8. The preparation method of the boric acid-containing polyvinyl alcohol nanoparticle of claim 4, further comprising, after the non-aqueous solvent is added to the polyvinyl alcohol aqueous solution and before the boric acid is added to the solution containing the polyvinyl alcohol nanoparticle, cooling the solution containing the polyvinyl alcohol nanoparticle to between 4° C. and 12° C.

9. The preparation method of the boric acid-containing polyvinyl alcohol nanoparticle of claim 4, wherein a concentration of the non-aqueous solvent is between 0.5 vol. % and 50 vol. %.

10. The preparation method of the boric acid-containing polyvinyl alcohol nanoparticle of claim 4, wherein a concentration of the polyvinyl alcohol aqueous solution is between 0.01 g/100 ml and 10 g/100 ml.
